(19) United States
(12) Patent Application Publication
(10) Pub. No.: US 2023/0360728 A1
(43) Pub. Date: Nov. 9, 2023

(54) BETA2- MICROGLOBULIN CONCENTRATION ANALYZING METHOD AND SYSTEM

(71) Applicant: INDUSTRIAL TECHNOLOGY RESEARCH INSTITUTE, Hsinchu (TW)

(72) Inventors: Ching-Hsuan CHANG, Taichung City (TW); Fang-Hsin LIN, Taichung City (TW); Kuan-Hung LIU, Taichung City (TW); Bin HSU, Tainan City (TW)

(73) Assignee: INDUSTRIAL TECHNOLOGY RESEARCH INSTITUTE, Hsinchu (TW)

(21) Appl. No.: 18/132,341

(22) Filed: Apr. 7, 2023

Related U.S. Application Data
(63) Continuation-in-part of application No. 18/096,447, filed on Jan. 12, 2023.
(60) Provisional application No. 63/329,043, filed on Apr. 8, 2022.
(60) Provisional application No. 63/329,043, filed on Apr. 8, 2022.

(30) Foreign Application Priority Data
Aug. 26, 2022 (TW) 111132190
Mar. 31, 2023 (TW) 112112459

(51) Int. Cl. G16B 25/10

(52) U.S. Cl. CPC G16B 25/10

(57) ABSTRACT

A beta2-microglobulin concentration analyzing method includes: inputting samples that are prepared from spent dialysate and corresponding to sampling time points into a differential mobility analyzing device for analysis to obtain beta2-microglobulin concentrations, multiplying the sampling time points with a dialysate flowrate respectively to obtain spent dialysate volumes, performing fitting on the beta2-microglobulin concentrations to obtain a concentration-spent dialysate volume fitting curve, and performing integration on the concentration-spent dialysate volume fitting curve to generate a total dialyzed weight.

### CROSS-REFERENCE TO RELATED APPLICATIONS

This non-provisional application claims priority under 35 U.S.C. § 119(a) on Patent Application No(s). 63/329,043 filed in the U.S. on Apr. 8, 2022, Patent Application No(s). 111132190 filed in Republic of China (ROC) on Aug. 26, 2022, patent application Ser. No. 18/096,447 filed in the U.S. on Jan. 12, 2023, and Patent Application No(s). 112112459 filed in Republic of China (ROC) on Mar. 31, 2023 the entire contents of which are hereby incorporated by reference.

### BACKGROUND

### 1. Technical Field

This disclosure relates to a beta2-microglobulin concentration analyzing method and system.

### 2. Related Art

Examination reports of patients with chronical renal disease often show excessive amount of uremic toxins accumulated in their body, and the concentration of beta2-microglobulin is regarded as one of the indicators for tracking concentration of middle-molecule uremic toxins in patients with chronical renal disease. Generally, labs use enzyme-linked immunosorbent assay (ELISA) to analyze the concentration of beta2-microglobulin in the specimen. However, the operation of ELISA requires intensive laboratory labor and long analysis time, and can't be used to determine the amount of uremic toxins is dialyzed out of the patient's body.

### SUMMARY

According to one or more embodiment of this disclosure, a beta2-microglobulin concentration analyzing method, performed by a processing device, includes: inputting a plurality of samples corresponding to a plurality of sampling time points into a differential mobility analyzing device for analysis to obtain a plurality of beta2-microglobulin concentrations, wherein the plurality of samples are prepared from spent dialysate; multiplying the plurality of sampling time points with a dialysate flowrate respectively to obtain a plurality of spent dialysate volumes; performing fitting on the plurality of beta2-microglobulin concentrations to obtain a concentration-spent dialysate volume fitting curve; and performing integration on the concentration-spent dialysate volume fitting curve to generate a total dialyzed weight.

According to one or more embodiment of this disclosure, a beta2-microglobulin concentration analyzing method, adapted to a differential mobility analyzing device and a processing device, wherein the differential mobility analyzing device includes an electrospray atomizer, a differential mobility analyzer and a condensation particle counter, and the method includes: inputting a plurality of samples corresponding to a plurality of sampling time points into the electrospray atomizer to obtain a plurality of aerosol samples, wherein the plurality of samples are prepared from spent dialysate; inputting the plurality of aerosol samples into the differential mobility analyzer to obtain a plurality of screened particle samples; inputting the plurality of screened particle samples into the condensation particle counter to obtain a plurality of particle counts; dividing, by the processing device, the plurality of particle counts by a volume of the dialysate entered per unit time respectively to obtain a plurality of beta2-microglobulin concentrations; multiplying, by the processing device, the plurality of sampling time points with a dialysate flowrate respectively to obtain a plurality of spent dialysate volumes; performing, by the processing device, fitting on the plurality of beta2-microglobulin concentrations to obtain a concentration-spent dialysate volume fitting curve; and performing, by the processing device, integration on the concentration-spent dialysate volume fitting curve to generate a total dialyzed weight.

According to one or more embodiment of this disclosure, a beta2-microglobulin concentration analyzing system includes: an electrospray atomizer, a differential mobility analyzer, a condensation particle counter and a processing device. The electrospray atomizer is configured to receive a plurality of samples corresponding to a plurality of sampling time points and output a plurality of aerosol samples, wherein the plurality of samples are prepared from spent dialysate. The differential mobility analyzer is configured to receive the plurality of aerosol samples and output a plurality of screened particle samples. The condensation particle counter configured to receive the plurality of screened particle samples and output a plurality of particle counts. The processing device is connected to the condensation particle counter and is configured to perform: dividing the plurality of particle counts by a volume of the dialysate entered per unit time respectively to obtain a plurality of beta2-microglobulin concentrations; multiplying the plurality of sampling time points with a dialysate flowrate respectively to obtain a plurality of spent dialysate volumes; performing fitting on the plurality of beta2-microglobulin concentrations to obtain a concentration-spent dialysate volume fitting curve; and performing integration on the concentration-spent dialysate volume fitting curve to generate a total dialyzed weight.

### BRIEF DESCRIPTION OF THE DRAWINGS

The present disclosure will become more fully understood from the detailed description given hereinbelow and the accompanying drawings which are given by way of illustration only and thus are not limitative of the present disclosure and wherein:

FIG. 1 is a block diagram illustrating a beta2-microglobulin concentration analyzing system according to an embodiment of the present disclosure;

FIG. 2 is a block diagram illustrating a beta2-microglobulin concentration analyzing system according to another embodiment of the present disclosure;

FIG. 3 is a block diagram illustrating a beta2-microglobulin concentration analyzing system according to yet another embodiment of the present disclosure;

FIG. 4 is a flowchart illustrating a beta2-microglobulin concentration analyzing method according to an embodiment of the present disclosure;

FIG. 5 is a detailed flowchart illustrating step S1 of FIG. 4;

FIG. 6 is a detailed flowchart illustrating step S13 of FIG. 5 according to an embodiment of the present disclosure;

FIG. 7 is a schematic diagram illustrating a purification procedure;

FIG. 8 is a schematic diagram illustrating a beta2-microglobulin concentration analyzing device according to an embodiment of the present disclosure;

FIG. 9 is a diagram illustrating a beta2-microglobulin concentration analyzing method according to an embodiment of the present disclosure;

FIG. 10(a) shows a test result of reference beta2-microglobulin concentration of commercial sample, FIG. 10(b) shows a test result of beta2-microglobulin concentration according to one or more embodiments of the present disclosure;

FIG. 11(a) shows a result of beta2-microglobulin concentration of 16 samples using ELISA, FIG. 11(b) shows a result of beta2-microglobulin concentration according to one or more embodiments of the present disclosure;

FIG. 12 is a block diagram illustrating a beta2-microglobulin concentration analyzing system according to still another embodiment of the present disclosure;

FIG. 13 is a flowchart illustrating a beta2-microglobulin concentration analyzing method according to another embodiment of the present disclosure;

FIG. 14(a), FIG. 14(b) and FIG. 14(c) are schematic diagrams respectively illustrating performing fitting on the beta2-microglobulin concentrations to obtain a concentration-spent dialysate volume fitting curve;

FIG. 15 is a flowchart illustrating a method of obtaining beta2-microglobulin concentrations according to an embodiment of the present disclosure;

FIG. 16(a) and FIG. 16(b) are schematic diagrams illustrating measurement data of beta2-microglobulin concentrations;

FIG. 17 is a schematic diagram of a concentration-spent dialysate volume fitting curve;

FIG. 18 is a flowchart illustrating a method of obtaining beta2-microglobulin concentrations according to another embodiment of the present disclosure; and

FIG. 19(a) is bar graph of a total dialyzed weight obtained by the beta2-microglobulin concentration analyzing method and system according to one or more embodiments of the present disclosure, and FIG. 19(b) is bar graph of a total dialyzed weight obtained by using prior art.

### DETAILED DESCRIPTION

In the following detailed description, for purposes of explanation, numerous specific details are set forth in order to provide a thorough understanding of the disclosed embodiments. According to the description, claims and the drawings disclosed in the specification, one skilled in the art may easily understand the concepts and features of the present invention. The following embodiments further illustrate various aspects of the present invention, but are not meant to limit the scope of the present invention.

Please refer to FIG. 1, wherein FIG. 1 is a block diagram illustrating a beta2-microglobulin concentration analyzing system according to an embodiment of the present disclosure. The beta2-microglobulin concentration analyzing system 1a includes a preparation device 11 and a differential mobility analyzing device 12. The preparation device 11 is configured to prepare a sample, the differential mobility analyzing device 12 is configured to analyze a concentration of the sample, wherein a process of transferring the sample prepared by the preparation device 11 to the differential mobility analyzing device 12 for analysis may be performed by laboratory personnel.

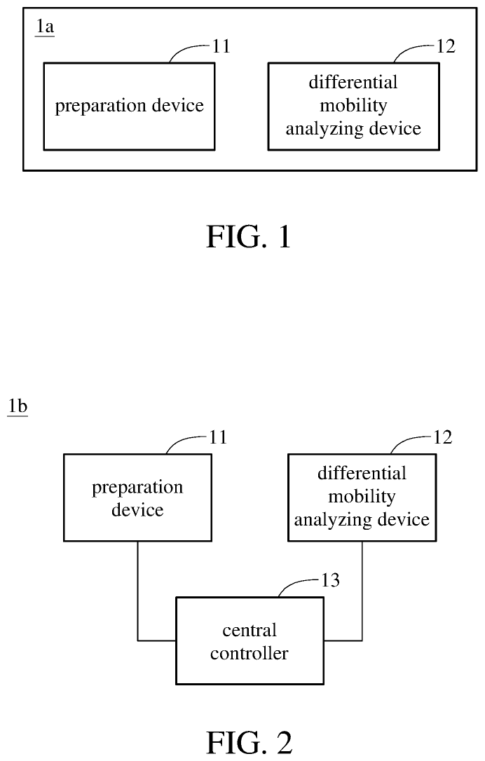

Please refer to FIG. 2, wherein FIG. 2 is a block diagram illustrating a beta2-microglobulin concentration analyzing system according to another embodiment of the present disclosure. The beta2-microglobulin concentration analyzing system 1b shown in FIG. 2 includes the preparation device 11, the differential mobility analyzing device 12 and a central controller 13. The central controller 13 is electrically connected to or in communication connection with the preparation device 11 and the differential mobility analyzing device 12, wherein the preparation device 11 and the differential mobility analyzing device 12 of the beta2-microglobulin concentration analyzing system 1b may be the same as the preparation device 11 and the differential mobility analyzing device 12 shown in FIG. 1, and description thereof are not repeated herein. The central controller 13 is, for example, a computer. The central controller 13 may output a notification after the preparation device 11 finishing the preparation of the sample, to notify laboratory personnel to transfer the sample to the differential mobility analyzing device 12. After the differential mobility analyzing device 12 receives the sample, the central controller 13 may control the differential mobility analyzing device 12 to perform analysis on the sample. Specifically, methods of the central controller 13 determining that the differential mobility analyzing device 12 has received the sample may include: determining that the differential mobility analyzing device 12 has received the sample based on a confirmation command that laboratory personnel inputted to the central controller 13; and/or the central controller 13 is connected to a sensing element (for example, a pressure sensor, a RFID sensor etc.) of a latching member of the differential mobility analyzing device 12, and the central controller 13 determining that the differential mobility analyzing device 12 has received the sample when the sensing element is triggered.

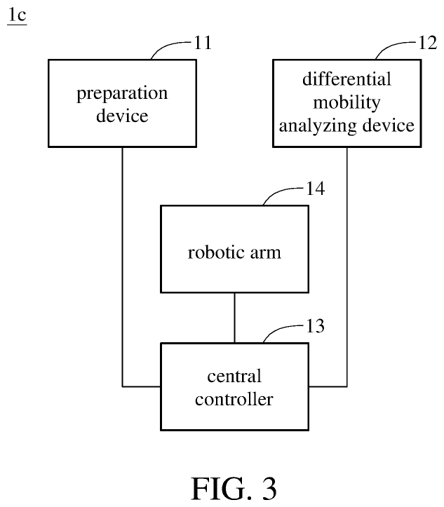

Please refer to FIG. 3, wherein FIG. 3 is a block diagram illustrating a beta2-microglobulin concentration analyzing system according to yet another embodiment of the present disclosure. The beta2-microglobulin concentration analyzing system 1c shown in FIG. 3 includes the preparation device 11, the differential mobility analyzing device 12, the central controller 13 and a robotic arm 14. The central controller 13 is electrically connected to or in communication connection with the preparation device 11, the differential mobility analyzing device 12 and the robotic arm 14, wherein the preparation device 11, the differential mobility analyzing device 12 and the central controller 13 of the beta2-microglobulin concentration analyzing system 1c may be the same as the preparation device 11, the differential mobility analyzing device 12 and the central controller 13 shown in FIG. 2, and description thereof are not repeated herein. The central controller 13 may be configured to control movement of the robotic arm 14, to control the robotic arm 14 to transfer a centrifuge tube loaded with dialysate to the preparation device 11, the central controller 13 may monitor the process of preparing the sample, and control the robotic arm 14 to transfer the sample to the differential mobility analyzing device 12 after the preparation of the sample is finished.

The beta2-microglobulin concentration analyzing systems shown in FIG. 1-FIG. 3 may be configured to prepare the sample with the dialysate, and configured to analyze the beta2-microglobulin concentration of the sample.

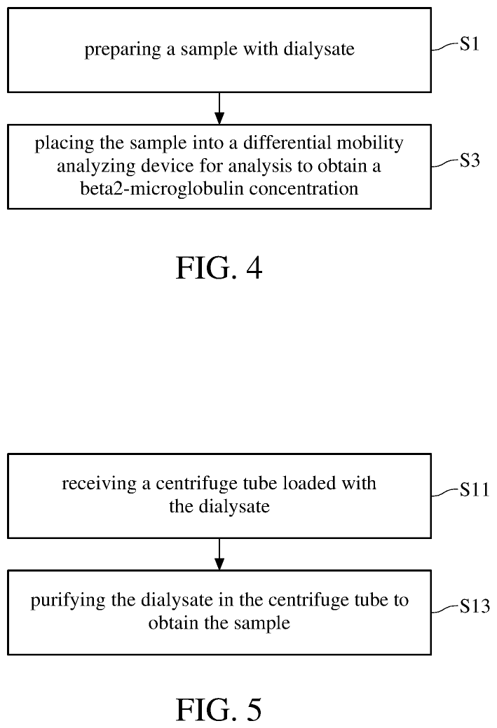

Please refer to FIG. 1 and FIG. 4, wherein FIG. 4 is a flowchart illustrating a beta2-microglobulin concentration analyzing method according to an embodiment of the present disclosure. As shown in FIG. 4, the beta2-microglobulin concentration analyzing method includes: step S1: preparing a sample with spent dialysate; and step S3: placing the sample into a differential mobility analyzing device for analysis to obtain a beta2-microglobulin concentration.

The preparation device 11 may include, for example, a centrifuge machine for filtering out impurity in the spent dialysate in step S1 to obtain the sample. In step S3, the laboratory personnel places the sample into the differential mobility analyzing device 12 for analysis to obtain the beta2-microglobulin concentration. Accordingly, the beta2-microglobulin concentration of the spent dialysate may be obtained with lower costs.

To explain the method of the preparation device 11 preparing the sample in more detail, please refer to FIG. 1 and FIG. 5, wherein FIG. 5 is a detailed flowchart illustrating step S1 of FIG. 4. As shown in FIG. 5, step S1 of FIG. 4 may include: step S11: receiving a centrifuge tube loaded with the spent dialysate; and step S13: purifying the spent dialysate in the centrifuge tube to obtain the sample.

In step S11, the laboratory personnel loads the spent dialysate into the centrifuge tube, and places the centrifuge tube into the preparation device 11 (for example, the centrifuge machine). In step S13, the laboratory personnel operates the preparation device 11 to centrifuge the centrifuge tube loaded with the spent dialysate to remove impurity from the spent dialysate, thereby obtaining the sample for analyzing the beta2-microglobulin concentration.

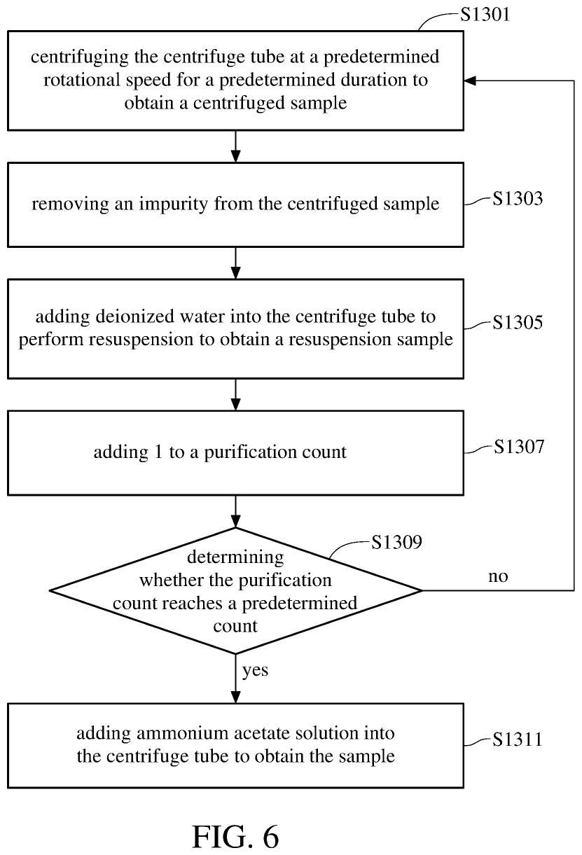

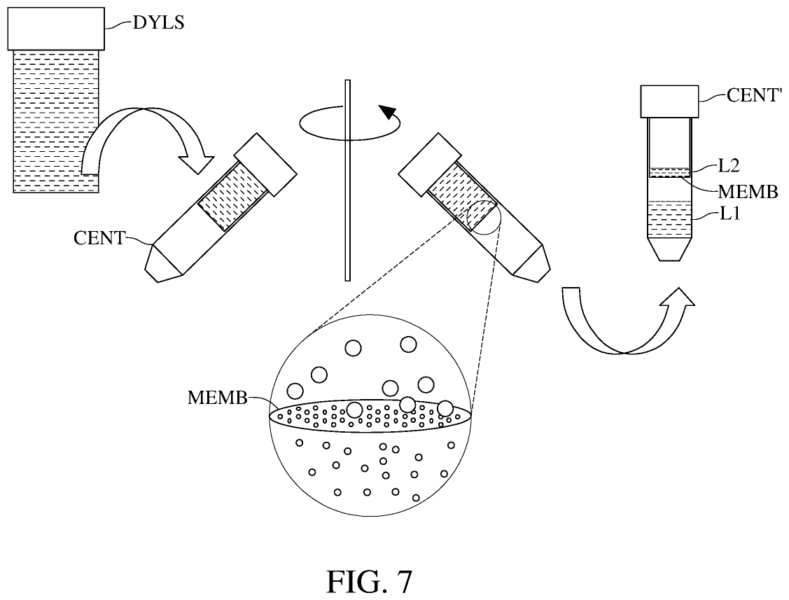

To further explain method of the preparation device 11 preparing the sample, and method of purifying the spent dialysate loaded in the centrifuge tube, please refer to FIG. 3, FIG. 6 and FIG. 7, wherein FIG. 6 is a detailed flowchart illustrating step S13 of FIG. 5 according to an embodiment of the present disclosure, and FIG. 7 is a schematic diagram illustrating a purification procedure. As shown in FIG. 6, step S13 of FIG. 5 may include: step S1301: centrifuging the centrifuge tube at a predetermined rotational speed for a predetermined duration to obtain a centrifuged sample; step S1303: removing an impurity from the centrifuged sample; step S1305: adding deionized water into the centrifuge tube to perform resuspension to obtain a resuspension sample; step S1307: adding 1 to a purification count; step S1309: determining whether the purification count reaches a predetermined count; if the determination result of step S1309 is “yes”, performing step S1311: adding ammonium acetate solution into the centrifuge tube to obtain the sample; and if the determination result of step S1309 is “no”, adding the deionized water into the centrifuge tube again to perform resuspension to obtain another resuspension sample, and performing step S1301 on the another resuspension sample. The purification procedure may include step S1301 to S1309 shown in FIG. 6.

The predetermined rotational speed is, for example, fourteen thousand rotations per minute (14 krpm), and the predetermined duration is, for example, 15 minutes. In step S1301, the laboratory personnel may operate the preparation device 11 to perform centrifugation on the centrifuge tube CENT loaded with the dialysate DYLS at the predetermined rotational speed, wherein the centrifuge tube CENT may include a filtration membrane MEMB, and molecular weight cut off (MWCO) of the filtration membrane MEMB may be 3 kD.

After centrifugation, the centrifuged sample in the centrifuge tube CENT′ may have layers, including an impurity layer L1 and a filtered layer L2 above the filtration membrane MEMB. The impurity layer L1 is the part that needs to be removed, and the filtered layer L2 is the part that needs to be kept. In step S1303, the laboratory personnel may use pipette to remove the impurity layer L1 from the centrifuged sample.

Then, in step S1305, the laboratory personnel adds deionized water into the centrifuged sample where impurity is already removed, to perform resuspension on the centrifuged sample with added deionized water to obtain the resuspension sample. The volume of the deionized water added into the centrifuge tube may be 500 μL. In step S1307, the central controller 13 may count a purification count. That is, the central controller 13 may add 1 to the purification count to record a number of times of performing resuspension, wherein an initial value of the purification count may be 0.

In step S1309, the laboratory personnel may determine whether the purification count reaches the predetermined count, wherein the predetermined count is, for example, 5. If the purification count reaches the predetermined count, the laboratory personnel performs step S1311, to add the ammonium acetate solution into the centrifuged sample containing deionized water, wherein a concentration of the ammonium acetate solution may be 20 mM. If the purification count does not reach the predetermined count, the laboratory personnel adds the deionized water into the centrifuge tube again to perform resuspension to obtain another resuspension sample, and performs the purification procedure on the another resuspension sample. Said “reach” herein refers to a situation of being equal to or larger than.

In short, step S1301 to step S1305 may be performed repeatedly for multiple times, wherein said multiple times equals to the predetermined count. When a number of times of performing step S1301 to step S1305 reaches the predetermined count, the laboratory personnel adds the ammonium acetate solution into the resuspension sample, to obtain the sample used for analyzing the beta2-microglobulin concentration, and the impurity concentration of the sample at this stage is less than 50 ppm.

It should be noted that the present disclosure does not limit the order of performing step S1305, S1307 and S1309, as long as step S1307 is performed prior to step S1309. In addition, numerical values of the predetermined rotational speed, the predetermined duration, volume of the deionized water, the purification count, the predetermined count and concentration of the ammonium acetate solution described in the embodiment of FIG. 6 is merely an example, the present disclosure does not limit the actual numerical values of these parameters.

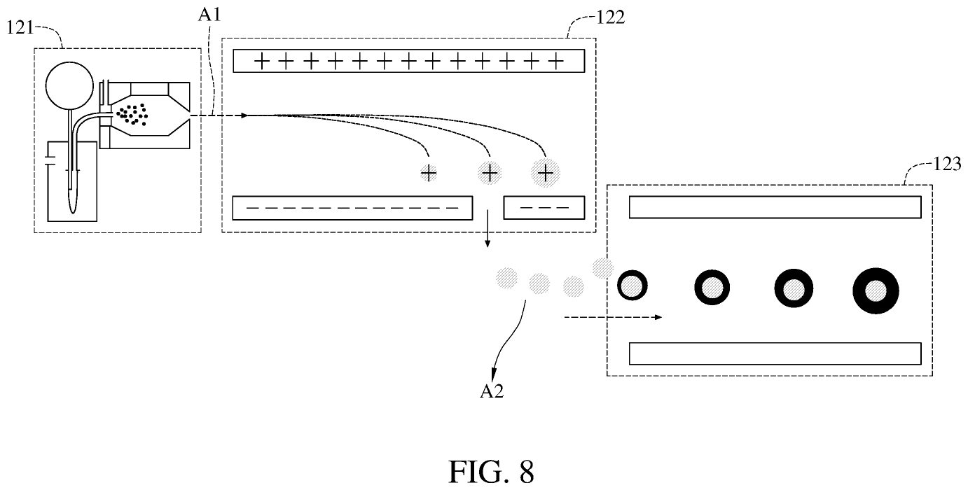

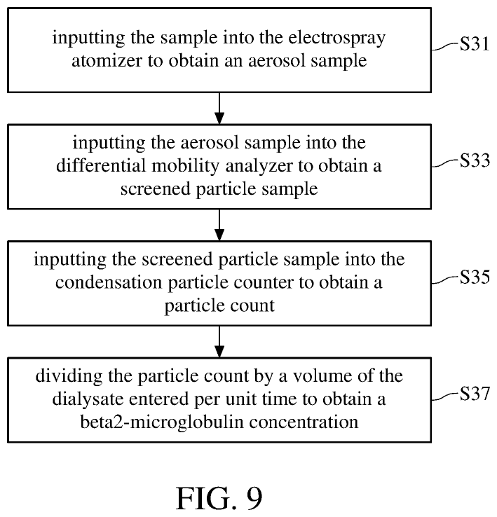

Please refer to FIG. 3, FIG. 8 and FIG. 9, wherein FIG. 8 is a schematic diagram illustrating a beta2-microglobulin concentration analyzing device according to an embodiment of the present disclosure, and FIG. 9 is a diagram illustrating a beta2-microglobulin concentration analyzing method according to an embodiment of the present disclosure. The differential mobility analyzing device 12 shown in FIG. 8 may be used to implement the differential mobility analyzing device 12 shown in FIG. 1 to FIG. 3, and the steps shown in FIG. 9 may be used to implement step S3 in FIG. 4.

As shown in FIG. 8, the differential mobility analyzing device 12 according to an embodiment of the present disclosure may include an electrospray atomizer 121, a differential mobility analyzer 122 and a condensation particle counter (CPC) 123. For example, the model number of the electrospray atomizer 121 may be TSI-3480; the model number of the differential mobility analyzer 122 may be TSI-3080; and the model number of the condensation particle counter 123 may be TSI-3776, but the present disclosure is not limited thereto.

The sample outlet of the electrospray atomizer 121 may be connected to the sample inlet of the differential mobility analyzer 122, and the sample outlet of the differential mobility analyzer 122 may be connected to the sample inlet of the condensation particle counter 123. Alternatively, the laboratory personnel or the robotic arm may transfer the sample produced by the electrospray atomizer 121 to the differential mobility analyzer 122 for analysis, and then transfer the sample produced by the differential mobility analyzer 122 to the condensation particle counter 123 for counting particle number.

As shown in FIG. 9, step S3 shown in FIG. 2 may include: step S31: inputting the sample into the electrospray atomizer to obtain an aerosol sample; step S33: inputting the aerosol sample into the differential mobility analyzer to obtain a screened particle sample; step S35: inputting the screened particle sample into the condensation particle counter to obtain a particle count; and step S37: dividing the particle count by a volume of the dialysate entered per unit time to obtain a beta2-microglobulin concentration.

In step S31, the electrospray atomizer 121 may receive the prepared sample from the preparation device 11, and apply a positive voltage to the sample for the electrosprayed liquid sample to produce positively charged aerosol sample A1, wherein an aerosol flowrate driven by the electrospray atomizer 121 may be 1.5 liters per minute (1.5 L/min), but the present disclosure does not limit the actual numerical value of the aerosol flowrate. In step S33, the laboratory personnel transfers the aerosol sample A1 from the electrospray atomizer 121 to the differential mobility analyzer 122, thereby selecting the screened particle sample A2 from the positively charged the aerosol sample A1. The screened particle sample A2 has a specified protein molecular weight and a specified protein size, and the specified protein molecular weight is, for example, 11.8 kD, and a range of the specified protein size is, for example, 3.85 nm to 4.14 nm. A sheath flowrate of the differential mobility analyzer 122 may be 20 liters per minute (20 L/min). The present disclosure does not limit the actual numerical values of the specified protein molecular weight, the specified protein size and the sheath flowrate.

In step S35, the laboratory personnel transfers the screened particle sample A2 from the differential mobility analyzer 122 to the condensation particle counter 123. The condensation particle counter 123 may be equipped with a laser optical detector, to calculate a number of particles with the specified protein molecular weight and the specified protein size through laser light. The laser wavelength of the condensation particle counter 123 may be 405 nm. The size of beta2-microglobulin (3.85 nm) is far smaller than a size range that is detectable by laser light. Therefore, general laser optical detector might not be suitable for beta2-microglobulin analysis of the present disclosure. The beta2-microglobulin screened by the differential mobility analyzer 122 should be condensed and grown on the particle surface to enlarge the size of beta2-microglobulin from 3.85 nm to micron level (>1 μm) before it can be used in the measurement and metering performed by the laser optical detector, which illustrates the necessity of using the condensation particle counter 123. In step S37, the laboratory personnel may divide the particle count of the screened particle sample A2 by the volume the spent dialysate loaded per unit time to obtain the beta2-microglobulin concentration. Step S37 may also be performed by the central controller 13.

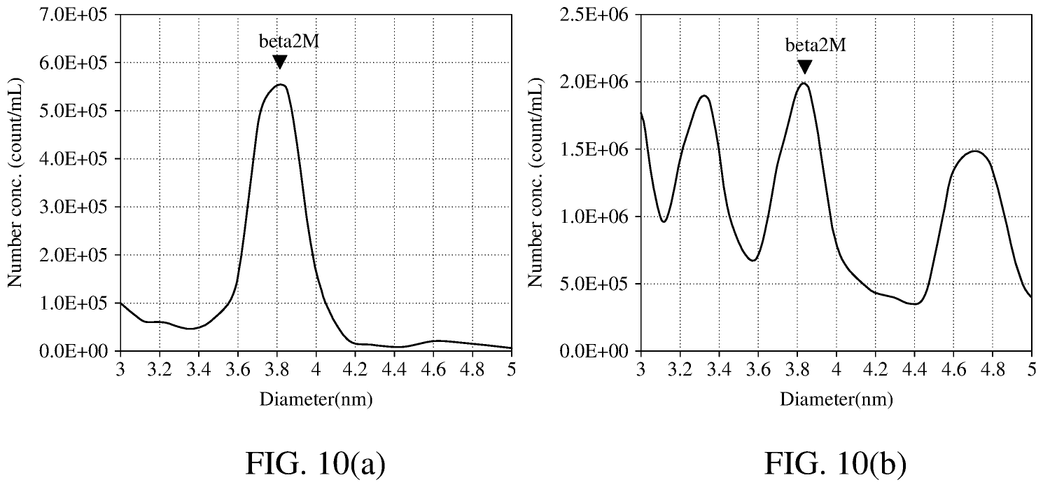

Please refer to FIG. 10(a) and FIG. 10(b), wherein FIG. 10(a) shows a test result of reference beta2-microglobulin concentration of commercial sample, FIG. 10(b) shows a test result of beta2-microglobulin concentration in the spent dialysate according to one or more embodiments of the present disclosure.

It can be seen from FIG. 10(a) that, the result of the beta2-microglobulin concentration of commercial sample indicates a maximum concentration value at around 3.85 nm (marked by “beta2M” in FIG. 10(a)), meaning the size (diameter) of beta2-microglobulin is around 3.85 nm. It can be seen from FIG. 10(b) that, the result of the beta2-microglobulin concentration obtained according to one or more embodiments of the present disclosure also shows a maximum concentration value at around 3.85 nm (marked by “beta2M” in FIG. 10(b)). Therefore, FIG. 10(a) and FIG. 10(b) show that the sample prepared by the beta2-microglobulin concentration analyzing method and system according to one or more embodiments of the present disclosure has sufficiently high beta2-microglobulin purity.

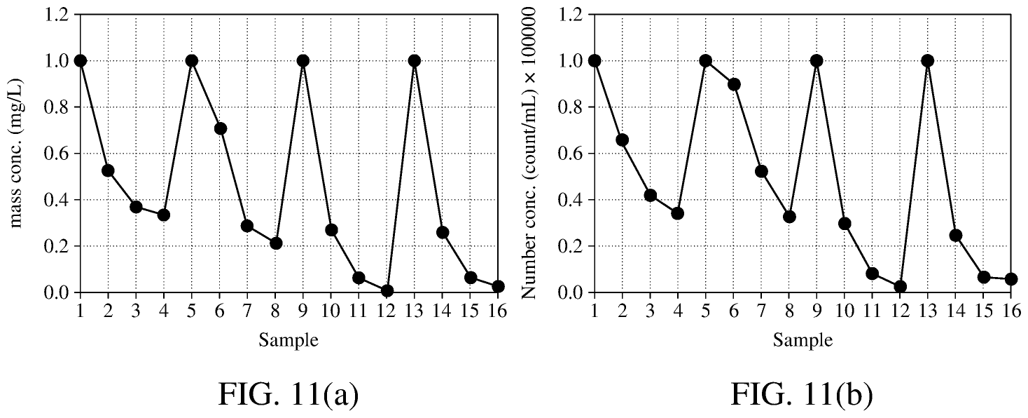

Please refer to FIG. 11(a) and FIG. 11(b), wherein FIG. 11(a) shows a result of beta2-microglobulin concentration of 16 samples using ELISA, FIG. 11(b) shows a result of beta2-microglobulin concentration according to one or more embodiments of the present disclosure, and FIG. 11(a) and FIG. 11(b) show test results of 16 samples prepared according to one or more embodiments of the present disclosure.

As seen from FIG. 11(a) and FIG. 11(b), the results obtained using ELISA and the results obtained using the beta2-microglobulin concentration analyzing method and system according to one or more embodiments of the present disclosure are in accordance with each other. Comparing to the beta2-microglobulin concentration analyzing method and system of the present disclosure, using ELISA for analysis requires additional antibodies and chemical substances. Therefore, FIG. 11(a) and FIG. 11(b) show that the beta2-microglobulin concentration analyzing method and system according to one or more embodiments of the present disclosure may be used to obtain an accurate result with lower cost.

It should be noted that the beta2-microglobulin concentration analyzing method according to one or more embodiments of the present disclosure are explained using the beta2-microglobulin concentration analyzing system 1a shown in FIG. 1, but said method may also be performed by the beta2-microglobulin concentration analyzing system 1b shown in FIG. 2 or the beta2-microglobulin concentration analyzing system 1c shown in FIG. 3. Specifically, in an embodiment where the method is performed by the beta2-microglobulin concentration analyzing system 1b, the central controller 13 may pre-store operating parameters (for example, the predetermined rotational speed and the predetermined duration) of the preparation device 11 and operating parameters (for example, the aerosol flowrate, the specified protein molecular weight, the specified protein size, the sheath flowrate and the laser wavelength) of the differential mobility analyzing device 12, so that the preparation device 11 and the differential mobility analyzing device 12 may process the sample according to the operating parameters after the laboratory personnel loads the sample into the preparation device 11 and the differential mobility analyzing device 12. The preparation device 11 and the differential mobility analyzing device 12 may output notification to a terminal device (for example, a computer, a mobile device etc.) at the laboratory personnel's end to notify the laboratory personnel to transfer the sample that has been processed and analyzed. In an embodiment where the method is performed by the beta2-microglobulin concentration analyzing system 1c, the central controller 13 may also pre-store operating parameters of the preparation device 11 and the differential mobility analyzing device 12, and the central controller 13 controls the robotic arm 14 to place the sample into the preparation device 11 and the differential mobility analyzing device 12, and controls the operation of the preparation device 11 and the differential mobility analyzing device 12 according to the operating parameters after the sample is placed into the preparation device 11 and the differential mobility analyzing device 12.

In other words, in the embodiment of FIG. 1, transferring the sample, removing impurity from the sample, controlling the operation of the preparation device 11 and the differential mobility analyzing device 12 and determining the purification count are performed by the laboratory personnel; in the embodiment of FIG. 2, transferring the sample and removing impurity from the sample are performed by the laboratory personnel, and determining the purification count and controlling the operation of the preparation device 11 and the differential mobility analyzing device 12 may be performed by the central controller 13; in the embodiment of FIG. 3, transferring the sample and removing impurity from the sample may be performed by the robotic arm 14 which is controlled by the central controller 13, and controlling the operation of the preparation device 11 and the differential mobility analyzing device 12 as well as determining the purification count may be performed by the central controller 13.

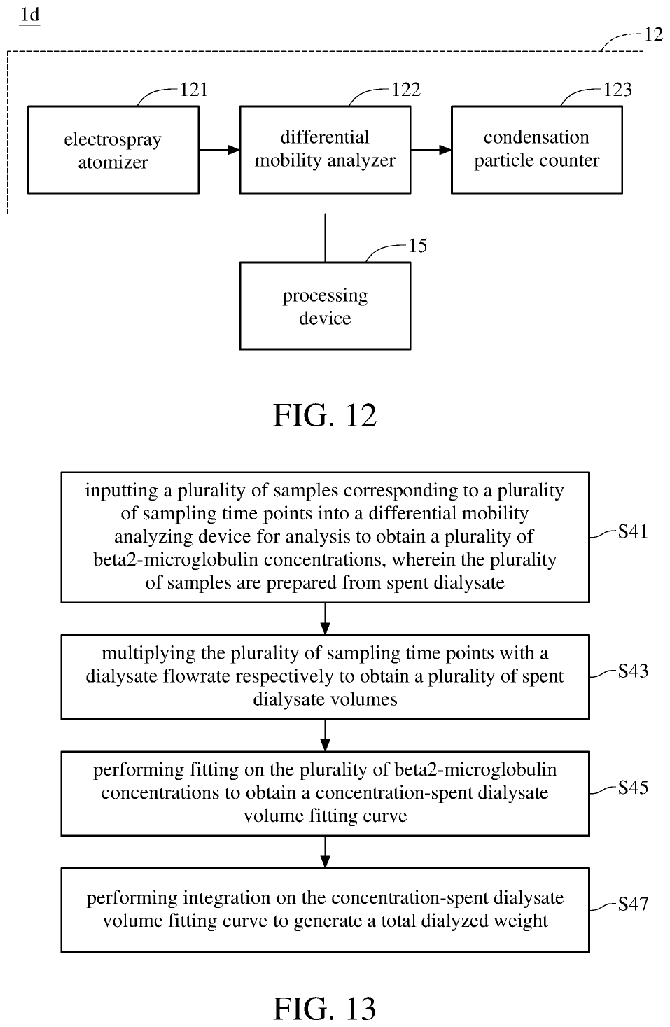

Please refer to FIG. 12, wherein FIG. 12 is a block diagram illustrating a beta2-microglobulin concentration analyzing system according to still another embodiment of the present disclosure. The beta2-microglobulin concentration analyzing system 1d includes the differential mobility analyzing device 12 and a processing device 15 connected to the differential mobility analyzing device 12. The differential mobility analyzing device 12 includes the electrospray atomizer 121, the differential mobility analyzer 122 and the condensation particle counter 123. The details of the differential mobility analyzing device 12 may be the same as the differential mobility analyzing device shown in FIGS. 1-3, and their repeated descriptions are omitted herein. The processing device 15 may include one or more processors, such as a central processing unit (CPU), a graphics processing unit (GPU), a microcontroller, a programmable logic controller (PLC) or other processors with signal processing function. In addition, the processing device 15 may also be the central controller 13 shown in FIGS. 1-3.

Please refer to FIG. 12 and FIG. 13, wherein FIG. 13 is a flowchart illustrating a beta2-microglobulin concentration analyzing method according to another embodiment of the present disclosure. As shown in FIG. 13, the beta2-microglobulin concentration analyzing method includes: step S41: inputting a plurality of samples corresponding to a plurality of sampling time points into a differential mobility analyzing device for analysis to obtain a plurality of beta2-microglobulin concentrations, wherein the plurality of samples are prepared from spent dialysate; step S43: multiplying the plurality of sampling time points with a dialysate flowrate respectively to obtain a plurality of spent dialysate volumes; step S45: performing fitting on the plurality of beta2-microglobulin concentrations to obtain a concentration-spent dialysate volume fitting curve; and step S47: performing integration on the concentration-spent dialysate volume fitting curve to generate a total dialyzed weight. Steps S43, S45 and S47 shown in FIG. 13 may be performed after step S3 of FIG. 4 and step S37 of FIG. 9.

In step S41, take the dialysis process of one subject as an example, the samples are obtained from the spent dialysate of different sampling time points during the dialysis process of the subject. In other words, one sample is at least a portion of the spent dialysate, one sample corresponds to one sampling time point, and the differential mobility analyzing device 12 performs analysis on the sample to generate one beta2-microglobulin concentration. The differential mobility analyzing device 12 may be the differential mobility analyzing device 12 shown in FIG. 8, and the method of using the differential mobility analyzing device 12 to obtain the beta2-microglobulin concentrations may be implemented with one or more embodiments shown in FIGS. 1 to 9, the details are not repeated herein.

In step S43, the processing device 15 multiplies each one of the sampling time points with the dialysate flowrate of the hemodialysis machine to obtain the spent dialysate volume of each one of the sampling time points. In other words, the spent dialysate volume refers to a volume of the dialysate already used at the corresponding sampling time point.

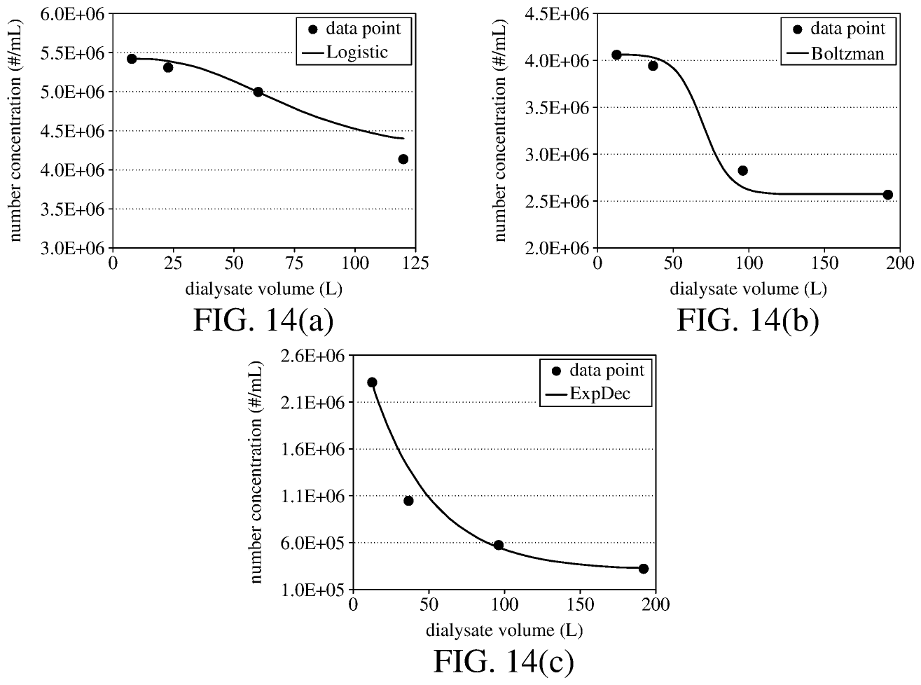

In step S45, the processing device 15 performs fitting on the beta2-microglobulin concentrations corresponding to the sampling time points respectively to obtain the concentration-spent dialysate volume fitting curve. Unit of the horizontal axis of the concentration-spent dialysate volume fitting curve generated in step S45 is, for example, the spent dialysate volume, and unit of the vertical axis of the concentration-spent dialysate volume fitting curve is, for example, a particle count per unit volume. The purpose of the fitting is to find a parameter with a smallest difference from the data point representing the beta2-microglobulin concentration. Said fitting may be a logistic fitting, a Boltzmann fitting or an exponential-decay fitting etc., wherein details of the fitting are described below in reference to FIG. 14(a) to FIG. 14(c).

In step S47, the processing device 15 performs integration on the concentration-spent dialysate volume fitting curve to calculate area under curve (AUC) of the concentration-spent dialysate volume fitting curve, and uses the AUC as the total dialyzed weight. The total dialyzed weight indicates the weight of the total amount of dialyzed beta2-microglobulin. Through the above embodiment, the cost and time required to calculate the total amount of dialyzed beta2-microglobulin of the dialysis process may be reduced, and medical personnel may check the amount of dialyzed uremic toxins of the patient.

Please refer to FIG. 14(a), wherein FIG. 14(a) is a schematic diagram illustrating performing the logistic fitting on the beta2-microglobulin concentrations to obtain a concentration-spent dialysate volume fitting curve. Step S45 shown in FIG. 13 may include using equation (1) to perform the logistic fitting on the beta2-microglobulin concentrations of four data points shown,

$$y = {A}_{2} + \frac{{A}_{1} - {A}_{2}}{1 + {( \frac{x}{{x}_{0}} )}^{p}} \qquad equation ( 1 )$$

wherein y is the beta2-microglobulin concentration corresponding to one sampling time point, and the unit of “y” is the number of particles per unit volume; x is the spent dialysate volume corresponding to the sampling time point; A₁ is a final permeable concentration; A₂ is an initial permeable concentration; x₀ is a spent dialysate volume for a default permeability; and p is a factor of the dialysate flowrate. The processing device 15 may perform logistic fitting using data analysis software of Origin.

A₁ is the final permeable concentration of the permeable membrane in the hemodialysis machine, and A₂ is the initial permeable concentration of the permeable membrane in the hemodialysis machine. Further, the initial permeable concentration indicates the beta2-microglobulin concentration that is permeable by the permeable membrane when the dialysis process has not yet begun or when the dialysis process has just begun. The initial permeable concentration may be considered as the filtration capacity in the initial stage of dialysis. The final permeable concentration indicates the beta2-microglobulin concentration that is permeable by the permeable membrane when the dialysis process ends or is about to end. The final permeable concentration may be considered as the filtration capacity in the final stage of dialysis. It should be noted that said “the dialysis process is about to end” may be set as a situation where the volume of spent dialysate has reached (equals to or exceeds) a preset volume and/or the dialysis time has reached (equals to or exceeds) preset time (for example, a preset time point, a present duration) etc., the present disclosure is not limited thereto.

x₀ indicates a volume of the spent dialysate at the default permeability of the permeable membrane. For example, assuming that the default permeability is 50%, then x₀ represents the volume of the spent dialysate when the permeability of the permeable membrane decreases to 50%. p is the factor of the dialysate flowrate and may be a coefficient generated by the data analysis software (Origin) as described above, and p may be considered as a decay rate of the beta2-microglobulin concentrations.

Please refer to FIG. 14(b), wherein 14(b) is a schematic diagram illustrating performing fitting on the beta2-microglobulin concentrations to obtain a concentration-spent dialysate volume fitting curve. Step S45 shown in FIG. 13 may include using equation (2) to perform Boltzman fitting on the beta2-microglobulin concentrations of four data points shown,

$$y = {A}_{2} + \frac{{A}_{1} - {A}_{2}}{1 + {e}^{( x - {x}_{0} ) / dx}} \qquad equation ( 2 )$$

wherein y is the beta2-microglobulin concentrations; x is the spent dialysate volume; A₁ is the final permeable concentration; A₂ is the initial permeable concentration; and x₀ is the spent dialysate volume for the default permeability. Definition of the parameters in equation (2) are the same as that of equation (1), their details are not repeated herein.

Please refer to FIG. 14(c), wherein 14(c) is a schematic diagram illustrating performing fitting on the beta2-microglobulin concentrations to obtain a concentration-spent dialysate volume fitting curve. Step S45 shown in FIG. 13 may include using equation (3) to perform exponential-decay fitting on the beta2-microglobulin concentrations of four data points shown,

y=A₂+A₁×e^{(-x/x}^{₀}⁾ equation (3)

wherein y is the beta2-microglobulin concentrations; x is the spent dialysate volume; A₁ is the final permeable concentration; A₂ is the initial permeable concentration; and x₀ is the spent dialysate volume for the default permeability. Definition of the parameters in equation (3) are the same as that of equation (1), their details are not repeated herein.

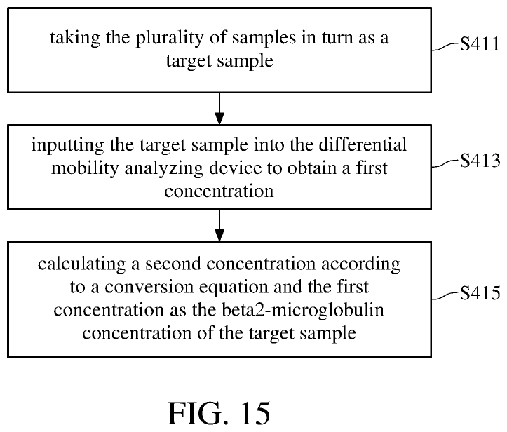

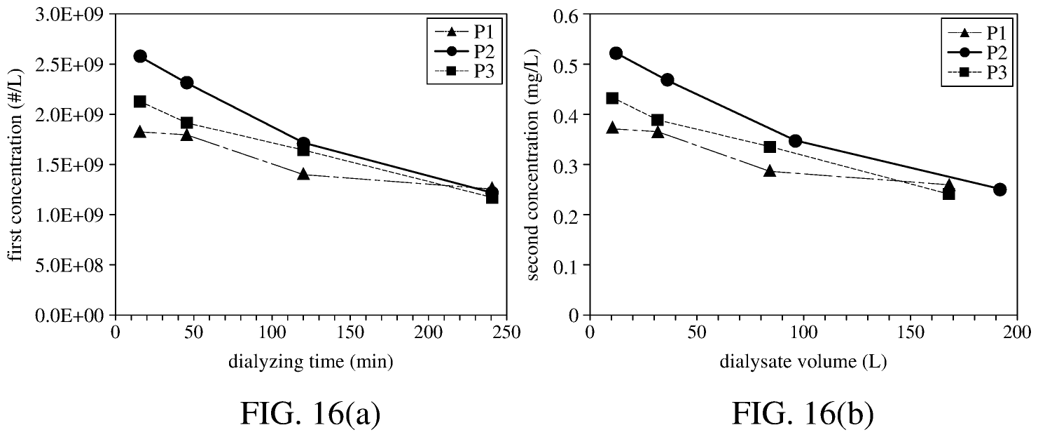

Please refer to FIG. 12, FIG. 15, FIG. 16(a) and FIG. 16(b), wherein FIG. 15 is a flowchart illustrating a method of obtaining beta2-microglobulin concentrations according to an embodiment of the present disclosure, FIG. 16(a) and FIG. 16(b) are schematic diagrams illustrating measurement data of beta2-microglobulin concentrations. Steps shown in FIG. 15 may be considered as a detail flowchart of an embodiment of step S41 shown in FIG. 13, and may be performed by the processing device 15 of FIG. 12. In FIG. 16(a) and FIG. 16(b), P1 to P3 each represents a subject, and details of the subjects P1 to P3 are shown in Table 1 below.

TABLE 1

| | P1 | P2 | P3 |
|---|---|---|---|
| dialysate flowrate (mL/min) | 700 | 800 | 700 |

| | |
|---|---|
| dialysis duration | spent dialysate volumes (L) |
| (sampling time points) | |

| | | | | |
|---|---|---|---|---|
| 15 | minutes | 10.5 | 12 | 10.5 |
| 45 | minutes | 31.5 | 36 | 31.5 |
| 120 | minutes | 84 | 96 | 84 |
| 240 | minutes | 168 | 192 | 168 |

As shown in FIG. 15, step S41 of FIG. 13 may include: step S411: taking the plurality of samples in turn as a target sample, and performing: step S413: inputting the target sample into the differential mobility analyzing device to obtain a first concentration; and step S415: calculating a second concentration according to a conversion equation and the first concentration as the beta2-microglobulin concentration of the target sample. The unit of the first concentration and the unit of the second concentration are different from each other. In short, in the embodiment of FIG. 15, the processing device 15 may perform unit conversion on the beta2-microglobulin concentration (the first concentration) corresponding to a first unit, to obtain the beta2-microglobulin concentration (the second concentration) corresponding to a second unit.

In step S411, the processing device 15 takes the samples corresponding to the sampling time points in turn as the target sample to perform step S413 and step S415 on the target sample.

In step S413, the processing device 15 may notify the laboratory personnel to transfer the target sample to the differential mobility analyzing device 12 or control the robotic arm 14 to transfer the target sample to the differential mobility analyzing device 12 as described above. The beta2-microglobulin concentration generated accordingly may be used as the first concentration. As shown in FIG. 16(a), the first concentration generated by the differential mobility analyzing device 12 is a particle count of beta2-microglobulin per liter.

In step S415, the processing device 15 calculates the second concentration according to the conversion equation and the first concentration and uses the second concentration as the beta2-microglobulin concentration of the target sample. The conversion equation may be equation (4) shown below,

C2=m×C1+DF equation (4)

wherein C2 is the second concentration; C1 is the first concentration; m is a slope of a linear relationship between the first concentration and the second concentration; DF is an intercept of the linear relationship between the first concentration and the second concentration. Equation (4) is a concentration relationship using two analysis methods to measure the beta2-microglobulin concentrations of multiple samples. The first concentration is a measured value using the differential mobility analyzing device; the second concentration is a measured value using ELISA; the parameter m is, for example, 2×10⁻¹⁰; and the parameter DF is, for example, 0.0071. The parameters m and DF may be adjusted according to requirements, the present disclosure is not limited thereto.

Then, the processing device 15 performs step S43 of FIG. 13 to obtain the dialysis duration (sampling time points) in FIG. 16(a) corresponding to the spent dialysate volumes, thereby generating the schematic diagram of FIG. 16(b).

As shown in FIG. 16(b), the unit of the second concentration generated by equation (4) is a weight (milligram) of beta2-microglobulin per liter of the spent dialysate. Please refer to Table 2 below, wherein the unit “#/L” is the particle count of beta2-microglobulin per liter, i.e. the first concentration; the unit “mg/L” is the weight (milligram) of beta2-microglobulin per liter of the spent dialysate, i.e. the second concentration. Table 2 shows the first concentration (#/L) and the second concentration (mg/L) after unit conversion.

TABLE 2

| | P1 | | P2 | | P3 | |
|---|---|---|---|---|---|---|
| | #/L | mg/L | #/L | mg/L | #/L | mg/L |
| 15 minutes | 1.83 × 10⁹ | 0.373 | 2.58 × 10⁹ | 0.523 | 2.13 × 10⁹ | 0.434 |
| 45 minutes | 1.79 × 10⁹ | 0.365 | 2.31 × 10⁹ | 0.470 | 1.92 × 10⁹ | 0.391 |
| 120 minutes | 1.40 × 10⁹ | 0.287 | 1.71 × 10⁹ | 0.349 | 1.65 × 10⁹ | 0.336 |
| 240 minutes | 1.26 × 10⁹ | 0.259 | 1.22 × 10⁹ | 0.252 | 1.17 × 10⁹ | 0.242 |

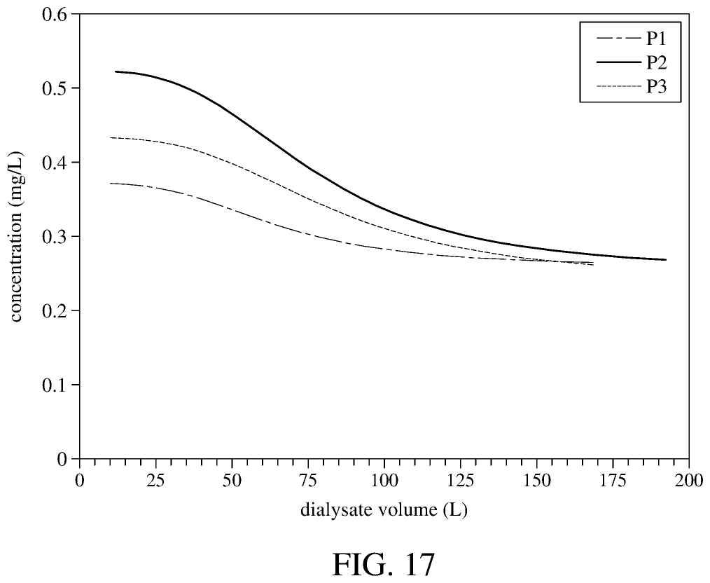

Please refer to FIG. 17, wherein FIG. 17 is a schematic diagram of a concentration-spent dialysate volume fitting curve. As shown in FIG. 17 after performing the fitting and unit conversion according to equation (4) on the data points of the beta2-microglobulin concentrations of each one of the subjects P1 to P3, the result shown in FIG. 17 may be obtained. The total dialyzed weight as shown in FIG. 17 may be shown in Table 3 below.

TABLE 3

| | P1 | P2 | P3 |
|---|---|---|---|
| total dialyzed | 51.8 | 71.9 | 57.9 |
| weight (milligram) | | | |

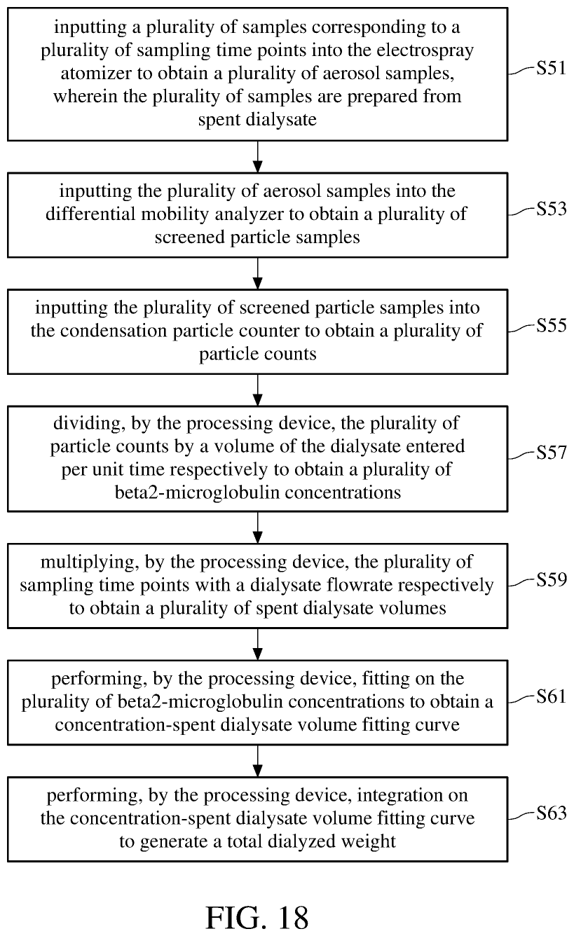

Please refer to FIG. 12 and FIG. 18, wherein FIG. 18 is a flowchart illustrating a method of obtaining beta2-microglobulin concentrations according to another embodiment of the present disclosure. Steps shown in FIG. 18 are adapted to the differential mobility analyzing device 12 and the processing device 15 shown in FIG. 12. As shown in FIG. 18, the beta2-microglobulin concentration analyzing method includes: step S51: inputting a plurality of samples corresponding to a plurality of sampling time points into the electrospray atomizer to obtain a plurality of aerosol samples, wherein the plurality of samples are prepared from spent dialysate; step S53: inputting the plurality of aerosol samples into the differential mobility analyzer to obtain a plurality of screened particle samples; step S55: inputting the plurality of screened particle samples into the condensation particle counter to obtain a plurality of particle counts; step S57: dividing, by the processing device, the plurality of particle counts by a volume of the dialysate entered per unit time respectively to obtain a plurality of beta2-microglobulin concentrations; step S59: multiplying, by the processing device, the plurality of sampling time points with a dialysate flowrate respectively to obtain a plurality of spent dialysate volumes; step S61: performing, by the processing device, fitting on the plurality of beta2-microglobulin concentrations to obtain a concentration-spent dialysate volume fitting curve; and step S63: performing, by the processing device, integration on the concentration-spent dialysate volume fitting curve to generate a total dialyzed weight.

Steps S51, S53, S55 and S57 shown in FIG. 18 are similar to steps S31, S33, S35 and S37 shown in FIG. 9 respectively, the difference is that steps S51, S53, S55 and S57 shown in FIG. 18 are performed on multiple samples. Steps S411, S413 and S415 of FIG. 15 may be an embodiment of step S57 shown in FIG. 18. That is, a quotient of the particle count of the target sample divided by the volume of the dialysate entered per unit time is used as the first concentration in step S411 of FIG. 15. Steps S59, S61 and S63 shown in FIG. 18 are the same as steps S43, S45 and S47 shown in FIG. 13 respectively. The details of FIG. 18 are not repeated herein.

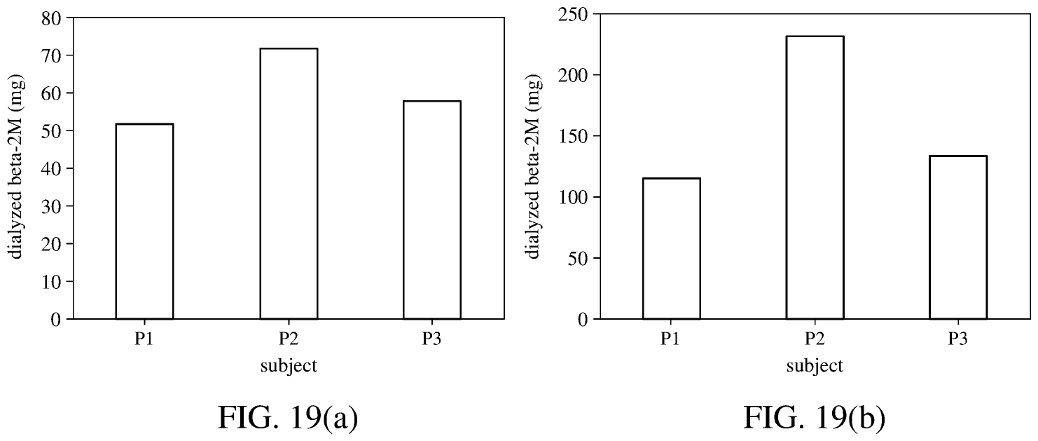

Please refer to FIG. 19(a) and FIG. 19(b), wherein FIG. 19(a) is bar graph of a total dialyzed weight obtained by the beta2-microglobulin concentration analyzing method and system according to one or more embodiments of the present disclosure, and FIG. 19(b) is bar graph of a total dialyzed weight obtained by using prior art. Said prior art uses immunoassay machine (Roche cobas c 502) to measure the beta2-microglobulin concentrations in the blood of the subjects before and after hemodialysis to calculate the total dialyzed weights of beta2-microglobulin. The calculation is as equation (5) shown below, and the results are as shown in Table 4 below,

$$β 2 M = ( \frac{β 2 {M}_{p r e - Hemo} \times B {V}_{p r e - Hemo}}{B {V}_{p r e - Hemo} - UF} - β 2 {M}_{post - Hemo} ) \times B {V}_{post} \qquad equation ( 5 )$$

wherein β2M is the total dialyzed weight of beta2-microglobulin; β2M_{pre-Hemo} is the beta2-microglobulin concentration in the blood of the subject before hemodialysis; β2M_{post-Remo} is the beta2-microglobulin concentration in the blood of the subject after hemodialysis; BV_{pre-Hemo} is a volume of blood of the subject before hemodialysis; BV_{post-Hemo} is a volume of blood of the subject after hemodialysis; and UF is a ultrafiltration volume.

TABLE 4

| | P1 | P2 | P3 |
|---|---|---|---|
| β2M_{pre-Hemo} (mg/L) | 23.1 | 41.9 | 36.4 |
| β2M_{post-Hemo} (mg/L) | 7.9 | 13.0 | 14.5 |
| BV_{pre-Hemo} (L) | 7.28 | 4.71 | 4.27 |
| BV_{post-Hemo} (L) | 7.26 | 4.59 | 4.20 |
| UF (L) | 0.2 | 1.6 | 0.9 |
| total dialyzed | 115.1 | 231.4 | 132.8 |
| weight (mg) | | | |

As seen from FIG. 19(a) and FIG. 19(b), the total dialyzed weights of beta2-microglobulin obtained by using the beta2-microglobulin concentration analyzing method and system according to one or more embodiments of the present disclosure show similar tendency as the total dialyzed weight of beta2-microglobulin obtained by using prior art. In addition, a correlation between the total dialyzed weights of beta2-microglobulin obtained by using the beta2-microglobulin concentration analyzing method and system according to one or more embodiments of the present disclosure and the total dialyzed weight of beta2-microglobulin obtained by using prior art is 0.987, which shows that the two are highly positive correlated.

In view of the above description, the beta2-microglobulin concentration analyzing method and system according to one or more embodiments of the present disclosure may lower the cost of analyzing the beta2-microglobulin concentration in the blood and lower time spent on sample preparation, processing and analysis, medical personnel may check the amount of dialyzed uremic toxins of the patient, and an accurate result may be obtained. In addition, the beta2-microglobulin concentration analyzing method and system according to one or more embodiments of the present disclosure may allow the particle count of screened beta2-microglobulin to be obtained by using the condensation particle counter.

What is claimed is:

1. A beta2-microglobulin concentration analyzing method, performed by a processing device, comprising:
inputting a plurality of samples corresponding to a plurality of sampling time points into a differential mobility analyzing device for analysis to obtain a plurality of beta2-microglobulin concentrations, wherein the plurality of samples are prepared from spent dialysate;
multiplying the plurality of sampling time points with a dialysate flowrate respectively to obtain a plurality of spent dialysate volumes;
performing fitting on the plurality of beta2-microglobulin concentrations to obtain a concentration-spent dialysate volume fitting curve; and
performing integration on the concentration-spent dialysate volume fitting curve to generate a total dialyzed weight.

2. The beta2-microglobulin concentration analyzing method according to claim 1, wherein performing the fitting on the plurality of beta2-microglobulin concentrations to obtain the concentration-spent dialysate volume fitting curve comprises: using equation (1) to perform logistic fitting on the plurality of beta2-microglobulin concentrations;
$$y = {A}_{2} + \frac{{A}_{1} - {A}_{2}}{1 + {( \frac{x}{{x}_{0}} )}^{p}} \qquad equation ( 1 )$$
wherein y is the plurality of beta2-microglobulin concentrations; x is the plurality of spent dialysate volumes; A₁ is a final permeable concentration; A₂ is an initial permeable concentration; x₀ is a dialysate volume for a default permeability; and p is a factor of the dialysate flowrate.

3. The beta2-microglobulin concentration analyzing method according to claim 1, wherein performing the fitting on the plurality of beta2-microglobulin concentrations to obtain the concentration-spent dialysate volume fitting curve comprises: using equation (1) to perform Boltzman fitting on the plurality of beta2-microglobulin concentrations;
$$y = {A}_{2} + \frac{{A}_{1} - {A}_{2}}{1 + {e}^{( x - {x}_{0} ) / dx}} \qquad equation ( 1 )$$
wherein y is the plurality of beta2-microglobulin concentrations; x is the plurality of spent dialysate volumes; A₁ is a final permeable concentration; A₂ is an initial permeable concentration; and x₀ is a spent dialysate volume for a default permeability.

4. The beta2-microglobulin concentration analyzing method according to claim 1, wherein performing the fitting on the plurality of beta2-microglobulin concentrations to obtain the concentration-spent dialysate volume fitting curve comprises: using equation (1) to perform exponential-decay fitting on the plurality of beta2-microglobulin concentrations;
y=A₂+A₁×e^{(-x/x}^{₀}⁾ equation (1)
wherein y is the plurality of beta2-microglobulin concentrations; x is the plurality of spent dialysate volumes; A₁ is a final permeable concentration; A₂ is an initial permeable concentration; and x₀ is a spent dialysate volume for a default permeability.

5. The beta2-microglobulin concentration analyzing method according to claim 1, wherein inputting the plurality of samples corresponding to the plurality of sampling time points into the differential mobility analyzing device for analysis to obtain the plurality of beta2-microglobulin concentrations comprises:
taking the plurality of samples in turn as a target sample, and performing:
inputting the target sample into the differential mobility analyzing device to obtain a first concentration; and
calculating a second concentration according to equation (1) and the first concentration as the beta2-microglobulin concentration of the target sample,
C2=m×C1+DF equation (1)
wherein C2 is the second concentration; C1 is the first concentration; m is a slope of a linear relationship between the first concentration and the second concentration; and DF is an intercept of the linear relationship between the first concentration and the second concentration.

6. A beta2-microglobulin concentration analyzing method, adapted to a differential mobility analyzing device and a processing device, wherein the differential mobility analyzing device comprises an electrospray atomizer, a differential mobility analyzer and a condensation particle counter, and the method comprises:
inputting a plurality of samples corresponding to a plurality of sampling time points into the electrospray atomizer to obtain a plurality of aerosol samples, wherein the plurality of samples are prepared from spent dialysate;
inputting the plurality of aerosol samples into the differential mobility analyzer to obtain a plurality of screened particle samples;
inputting the plurality of screened particle samples into the condensation particle counter to obtain a plurality of particle counts;
dividing, by the processing device, the plurality of particle counts by a volume of the dialysate entered per unit time respectively to obtain a plurality of beta2-microglobulin concentrations;
multiplying, by the processing device, the plurality of sampling time points with a dialysate flowrate respectively to obtain a plurality of spent dialysate volumes;
performing, by the processing device, fitting on the plurality of beta2-microglobulin concentrations to obtain a concentration-spent dialysate volume fitting curve; and
performing, by the processing device, integration on the concentration-spent dialysate volume fitting curve to generate a total dialyzed weight.

7. The beta2-microglobulin concentration analyzing method according to claim 6, wherein performing the fitting on the plurality of beta2-microglobulin concentrations to obtain the concentration-spent dialysate volume fitting curve comprises: using equation (1) to perform logistic fitting on the plurality of beta2-microglobulin concentrations;
$$y = {A}_{2} + \frac{{A}_{1} - {A}_{2}}{1 + {( \frac{x}{{x}_{0}} )}^{p}} \qquad equation ( 1 )$$
wherein y is the plurality of beta2-microglobulin concentrations; x is the plurality of spent dialysate volumes; A₁ is a final permeable concentration; A₂ is an initial permeable concentration; x₀ is a spent dialysate volume for a default permeability; and p is a factor of the dialysate flowrate.

8. The beta2-microglobulin concentration analyzing method according to claim 6, wherein performing the fitting on the plurality of beta2-microglobulin concentrations to obtain the concentration-spent dialysate volume fitting curve comprises: using equation (1) to perform Boltzman fitting on the plurality of beta2-microglobulin concentrations;
$$y = {A}_{2} + \frac{{A}_{1} - {A}_{2}}{1 + {e}^{( x - {x}_{0} ) / dx}} \qquad equation ( 1 )$$
wherein y is the plurality of beta2-microglobulin concentrations; x is the plurality of spent dialysate volumes; A₁ is a final permeable concentration; A₂ is an initial permeable concentration; and x₀ is a spent dialysate volume for a default permeability.

9. The beta2-microglobulin concentration analyzing method according to claim 6, wherein performing the fitting on the plurality of beta2-microglobulin concentrations to obtain the concentration-spent dialysate volume fitting curve comprises: using equation (1) to perform exponential-decay fitting on the plurality of beta2-microglobulin concentrations;
y=A₂+A₁×e^{(-x/x}^{₀}⁾ equation (1)
wherein y is the plurality of beta2-microglobulin concentrations; x is the plurality of spent dialysate volumes; A₁ is a final permeable concentration; A₂ is an initial permeable concentration; and x₀ is a spent dialysate volume for a default permeability.

10. The beta2-microglobulin concentration analyzing method according to claim 6, wherein dividing, by the processing device, the plurality of particle counts by the volume of the dialysate entered per unit time respectively to obtain the plurality of beta2-microglobulin concentrations comprises:
taking the plurality of samples in turn as a target sample, and performing:
using a quotient of the particle count divided by the volume of the dialysate entered per unit time as a first concentration; and
calculating a second concentration according to equation (1) and the first concentration as the beta2-microglobulin concentration of the target sample,
C2=m×C1+DF equation (1)
wherein C2 is the second concentration; C1 is the first concentration; m is a slope of a linear relationship between the first concentration and the second concentration; and DF is an intercept of the linear relationship between the first concentration and the second concentration.

11. A beta2-microglobulin concentration analyzing system, comprising:
an electrospray atomizer configured to receive a plurality of samples corresponding to a plurality of sampling time points and output a plurality of aerosol samples, wherein the plurality of samples are prepared from spent dialysate;
a differential mobility analyzer configured to receive the plurality of aerosol samples and output a plurality of screened particle samples;
a condensation particle counter configured to receive the plurality of screened particle samples and output a plurality of particle counts; and
a processing device connected to the condensation particle counter and configured to perform:
dividing the plurality of particle counts by a volume of the dialysate entered per unit time respectively to obtain a plurality of beta2-microglobulin concentrations;
multiplying the plurality of sampling time points with a dialysate flowrate respectively to obtain a plurality of spent dialysate volumes;
performing fitting on the plurality of beta2-microglobulin concentrations to obtain a concentration-spent dialysate volume fitting curve; and
performing integration on the concentration-spent dialysate volume fitting curve to generate a total dialyzed weight.

12. The beta2-microglobulin concentration analyzing system according to claim 11, wherein the processing device performing the fitting on the plurality of beta2-microglobulin concentrations to obtain the concentration-spent dialysate volume fitting curve comprises: using equation (1) to perform logistic fitting on the plurality of beta2-microglobulin concentrations;
$$y = {A}_{2} + \frac{{A}_{1} - {A}_{2}}{1 + {( \frac{x}{{x}_{0}} )}^{p}} \qquad equation ( 1 )$$
wherein y is the plurality of beta2-microglobulin concentrations; x is the plurality of spent dialysate volumes; A₁ is a final permeable concentration; A₂ is an initial permeable concentration; x₀ is a spent dialysate volume for a default permeability; and p is a factor of the dialysate flowrate.

13. The beta2-microglobulin concentration analyzing system according to claim 11, wherein the processing device performing the fitting on the plurality of beta2-microglobulin concentrations to obtain the concentration-spent dialysate volume fitting curve comprises: using equation (1) to perform Boltzman fitting on the plurality of beta2-microglobulin concentrations;
$$y = {A}_{2} + \frac{{A}_{1} - {A}_{2}}{1 + {e}^{( x - {x}_{0} ) / dx}} \qquad equation ( 1 )$$
wherein y is the plurality of beta2-microglobulin concentrations; x is the plurality of spent dialysate volumes; A₁ is a final permeable concentration; A₂ is an initial permeable concentration; and x₀ is a spent dialysate volume for a default permeability.

14. The beta2-microglobulin concentration analyzing system according to claim 11, wherein the processing device performing the fitting on the plurality of beta2-microglobulin concentrations to obtain the concentration-spent dialysate volume fitting curve comprises: using equation (1) to perform exponential-decay fitting on the plurality of beta2-microglobulin concentrations;
y=A₂+A₁×e^{(-x/x}^{₀}⁾ equation (1)
wherein y is the plurality of beta2-microglobulin concentrations; x is the plurality of spent dialysate volumes; A₁ is a final permeable concentration; A₂ is an initial permeable concentration; and x₀ is a spent dialysate volume for a default permeability.

15. The beta2-microglobulin concentration analyzing system according to claim 11, wherein the processing device performing dividing the plurality of particle counts by the volume of the dialysate entered per unit time respectively to obtain the plurality of beta2-microglobulin concentrations comprises:
taking the plurality of samples in turn as a target sample, and performing:
using a quotient of the particle count divided by the volume of the dialysate entered per unit time as a first concentration; and
calculating a second concentration according to equation (1) and the first concentration as the beta2-microglobulin concentration of the target sample,
C2=m×C1+DF equation (1)
wherein C2 is the second concentration; C1 is the first concentration; m is a slope of a linear relationship between the first concentration and the second concentration; and DF is an intercept of the linear relationship between the first concentration and the second concentration.
